(19) United States
(12) Patent Application Publication
(10) Pub. No.: US 2025/0288051 A1
(43) Pub. Date: Sep. 18, 2025

(54) SUPPORTIVE FOOTWEAR SYSTEMS

(71) Applicant: Mark Costin Roser, Hebron, CT (US)

(72) Inventor: Mark Costin Roser, Hebron, CT (US)

(21) Appl. No.: 18/860,439

(22) PCT Filed: Apr. 28, 2023
(86) PCT No.: PCT/US2023/020404

§ 371 Date: Oct. 25, 2024

Related U.S. Application Data
(60) Provisional application No. 63/335,729, filed on Apr. 28, 2022.

(51) Int. Cl. A43B 7/20; A43B 23/02

(52) U.S. Cl. CPC A43B 7/20; A43B 23/0245

(57) ABSTRACT

Embodiments disclosed herein relate to footwear systems and restorative energy systems configured to provide lateral support to a lower limb of a user when worn. A footwear system may include a footwear body, a yoke configured to rotate relative to the footwear body, an energy storage device configured to store and/or release energy, and one or more supports to provide lateral support. A restorative energy system may include a base and/or a yoke of an exoskeleton, an energy storage device similarly configured to store and/or release energy, and one or more supports to provide lateral support as well. In some embodiments, the supports may be interchangeable in the above systems to permit a controllable level of lateral support to be realized for a user.

### CROSS-REFERENCE TO RELATED APPLICATIONS

This application claims benefit under 35 U.S.C. § 119(e) of U.S. provisional application Ser. No. 63/335,729, filed Apr. 28, 2022, the disclosure of which is incorporated herein by reference in its entirety.

### FIELD

Disclosed embodiments are related to footwear which may include structural supports to aid the support and locomotion of a user's ankle.

### BACKGROUND

Footwear such as boots have multiple duties that they help people perform. These duties include protection from weather, uneven ground, and other environmental conditions as well as providing support for various portions of a user's foot and/or ankle. Separate prosthetics that may be attached to a user's leg such as ankle braces may be separately used to provide additional stability and ankle protection for a user during certain types of activities and/or after an injury.

### SUMMARY

In some embodiments, a footwear system is provided. The footwear system may include a footwear body and a yoke that is configured to rotate relative to at least a portion of the footwear body in a sagittal plane in dorsiflexion and plantarflexion. The footwear system may also include an energy storage device connected to the yoke and the portion of the footwear body. The footwear system may also include one or more supports configured to be engaged with the footwear body and/or the yoke. In some embodiments, the one or more supports are configured to provide lateral support to an ankle of a user when the footwear system is worn by the user.

In some embodiments, a method of using a footwear system is provided. The method may include rotating a yoke relative to a portion of a footwear body during dorsiflexion and plantarflexion of a user's ankle and storing and/or releasing energy with an energy storage device during the relative rotation of the yoke and the portion of the footwear to assist motion of the user's ankle. The method may also include engaging one or more supports with the footwear body and/or the yoke to provide lateral support to the user's ankle.

In some embodiments, a restorative energy system for footwear is provided. The system may include a base configured to be engaged with a first portion of a footwear body and a yoke configured to be engaged with a second portion of the footwear body, where the yoke may be configured to rotate relative to the base and the first portion of the footwear body. The system may also include an energy storage device connected to the base and the yoke as well as one or more supports which may be attached to the base and/or the yoke. In some embodiments, the one or more supports may be configured to provide lateral support to an ankle of the user.

In some embodiments, a method of using a restorative energy system for footwear is provided. The method may include engaging a base with a first portion of a footwear body and engaging a yoke to a second portion of the footwear body, where the yoke may be configured to rotate relative to the base and the first portion of the footwear body. The method may also include connecting an energy storage device to the base and the yoke and storing and/or releasing energy with the energy storage device during the rotation of the yoke relative to the base and the first portion of the footwear body. The method may also include engaging one or more supports with the base and/or the yoke to provide lateral support to an ankle of a user.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 presents a schematic view of a medial side of footwear having supports, according to some embodiments;

FIG. 2 presents a schematic view of FIG. 1A, where the supports are shown to be disposed in corresponding receptacles, according to some embodiments;

FIG. 3 presents a schematic view of a medial side of footwear having receptacles, according to some embodiments;

FIG. 4 presents a schematic view of a lateral side of footwear having receptacles, according to some embodiments;

FIG. 5A presents a schematic view of footwear having supports and an energy storage device, according to some embodiments;

FIG. 5B presents a schematic view of footwear having supports and an energy storage device, according to some embodiments;

FIG. 6A presents a schematic view of footwear having an exoskeleton attached to and bracing an upper region of the footwear, according to some embodiments;

FIG. 6B presents an enlarged view of region 6B of FIG. 6A;

FIG. 7A presents a schematic view of footwear having an exoskeleton attached to and bracing a lower region of the footwear, according to some embodiments;

FIG. 7B presents an enlarged view of region 7B of FIG. 7A;

FIG. 7C presents an enlarged rear view of region 7C of FIG. 7B;

FIG. 8A presents a schematic view of footwear having an exoskeleton attached to and bracing an upper and a lower region of the footwear, and further comprising supports, according to some embodiments;

FIG. 8B presents a schematic side and front view of the supports of FIG. 8A with a first size, according to some embodiments;

FIG. 8C presents a schematic side and front view of the supports of FIG. 8A with a second size, according to some embodiments;

FIG. 8D presents a schematic side and front view of the supports of FIG. 8A with a third size, according to some embodiments;

FIG. 9A presents a schematic view of a support configured for use in footwear, according to some embodiments;

FIG. 9B presents a schematic view of a support configured for use in footwear further comprising detents, according to some embodiments;

FIG. 10A presents a schematic side view of a support configured for use in footwear, according to some embodiments;

FIG. 10B presents a schematic side view of a support configured for use in footwear, according to some embodiments;

FIG. 11A presents a schematic rear view of footwear having an energy dissipation element, according to some embodiments;

FIG. 11B presents a schematic side view of FIG. 11A; and

FIG. 12 presents a schematic rear view of footwear having a lateral damper, according to some embodiments.

### DETAILED DESCRIPTION

While a variety of footwear may be worn by a user, such footwear may lack the support needed to ensure protection of a user's lower limb (e.g., foot, ankle, etc.) during certain activities and/or recovery from medical injuries. A lack of support in footwear may lead to a variety of lower limb injuries including, but not limited to ankle sprain, chronic ankle instability, Achilles tendon issues, plantar fasciitis, Charcot foot, stroke, idiopathic foot pain, partial amputation, tarsal tunnel, metatarsalgia, and many other medical issues. To treat and/or prevent many of these issues, it is important to employ footwear which includes components to facilitate structural support for a user's lower limb while the footwear is worn. In particular, providing lateral support to the lower limb of a user, and specifically to an ankle of the user in some embodiments, while the user is wearing footwear is desirable to ensuring stability and protection of the user's lower limb, and thus to treating and preventing the aforementioned medical issues.

While devices such as ankle braces have been employed to provide support for a user's lower limb, numerous different ankle braces having varying degrees of stiffness and support are often required to promote recovery of a lower limb injury of the user (e.g., an ankle sprain) as the user progresses through the recovery process. For example, three different stages of ankle braces may be required to be worn by the user for a given recovery process, with the first stage including a high stiffness brace, the second stage including a medium stiffness brace, and the third stage including an ankle wrapping brace having a low stiffness. The user may also be required to undergo a fourth stage involving the use of tape or a compression wrap to stabilize the lower limb of the user. Notably, the stiffness in each of these stages progressively decreases as the user advances through the injury recovery process. In addition to typically requiring multiple different braces to promote recovery, these braces or similar support devices may also not have a controllable level of support apart from the traditional high, medium, and low stiffness levels noted above. Even if other levels of stiffness are provided, it would require additional braces and recovery stages to be employed since these braces typically have a non-adjustable level of stiffness. These devices may also oftentimes be bulky and rigid, and thus be difficult to wear in combination with footwear which may prevent their use in certain situations as a user may be unable to wear appropriate footwear for an activity while using these braces.

In view of the above, the inventors have recognized benefits associated with employing a device and system for providing a controllable level of lateral support in footwear to provide a desired, and in some instances an adjustable, amount of support for a user's lower limb. The level of support may be selected depending on the application for which the device is used as well as the stage of injury that the user is in at the time of use of the device. For example, if the user is being treated for an early stage ankle sprain, a greater amount of lateral support may be employed immediately following injury to the affected limb(s), and then a subsequent reduction in the amount of lateral support may be provided as the user progresses through the recovery process. The inventors have found particular benefit with such controllable levels of lateral support being achieved through the use of a singular system, in some embodiments, rather than through employing multiple ankle braces or similar devices having different individual stiffnesses noted above. The ability to adjustably reduce support allows a non-affected limb to not receive unnecessary support when not needed and rapidly deploy support when approaching terrain with ankle/foot hazards.

Moreover, the inventors have recognized benefits associated with providing a controllable level of lateral support in conjunction with systems that aid in the locomotion of a user's foot and ankle. This may be achieved by providing the footwear devices according to embodiments disclosed herein in conjunction with energy storage devices that release and store energy to assist in the movement of a user's lower limb (e.g., foot and ankle) coincidentally with one or more stages of the gait cycle. In particular, to promote lateral support of a user's lower limb while engaging in the gait cycle, it is important for the device according to embodiments disclosed herein to also be able to promote propulsive support (e.g., plantarflexion and/or dorsiflexion of a user's lower limb including for example an ankle) and lower limb stability in the footwear (e.g., the ability to cradle the heel and other foot and/or ankle components while the device is in use).

The inventors have found that the aforementioned benefits may be realized by providing a footwear (e.g., a boot or high-top shoe) with an exoskeleton in some embodiments. In turn, the exoskeleton may include one or more receptacles that are configured to receive one or more supports, where the one or more supports are configured to alter the functional characteristics, such as the lateral support provided to an ankle, of the footwear. In addition or alternatively, the one or more supports may be engaged directly with the footwear without the use of a separate exoskeleton to provide a varying degree of support to the footwear. In any case, the one or more supports may be attached, inserted, affixed, fastened, or otherwise engaged to the footwear and/or the exoskeleton to promote lateral support in a frontal plane (e.g., to prevent against inversion and/or eversion) while also enabling propulsive support in a sagittal plane and lower limb stability in the footwear.

According to some aspects, a footwear system may include a restorative energy system formed using a portion of the footwear body and a yoke. In some embodiments, the yoke may be configured to rotate relative to at least a portion of the footwear body in a sagittal plane to promote dorsiflexion and plantarflexion of a user's lower limb in accordance with the stages of the gait cycle. In some embodiments, an energy storage device (e.g., a spring) may be provided and connected to the yoke and the portion of the footwear body (e.g., the base) to promote the storing and/or releasing of energy to assist with movement of the user's foot and ankle during the gait cycle. In some embodiments, one or more supports may also be provided and configured to be engaged with the footwear body and/or the yoke. The one or more supports may be configured to provide lateral support to an ankle of a user when the footwear system is worn by the user.

According to some aspects, a separate restorative energy system is provided and configured to be engaged with a corresponding article of footwear (e.g., a boot). The restorative energy system may include an exoskeleton frame having a base and a yoke. The exoskeleton system may be integrated with the footwear during manufacture of the footwear or in an aftermarket capacity, and the integration of the exoskeleton system may be such that the exoskeleton is positioned between layers of an article of footwear, internal to the footwear, external to the footwear, or a combination thereof. The base and the yoke of the footwear may be configured to be engaged with first and second portions of a footwear body, respectively. The yoke may also be configured to rotate relative to the base and the first portion of the footwear body to help promote propulsive support regarding plantarflexion and dorsiflexion of the user's foot and ankle. An energy storage device may also be provided and connected to the base and the yoke. For example, extension of the energy storage device may store energy during at least a portion of a range of movement of the yoke when the yoke is rotated in a first direction and the energy storage device may release energy during rotation in a second direction opposite the first direction. The energy storage device may be employed to promote the storing and/or releasing of energy to assist with movement of the user's foot and ankle in accordance with the gait cycle. In some embodiments, one or more supports may be provided and configured to be attached to the base and/or the yoke to provide lateral support to an ankle of a user. Without wishing to be bound by theory, in some embodiments, only one of either the base and/or yoke may be provided in a footwear system or restorative energy system. In other embodiments, however, both of the base and the yoke may be provided, or neither the base nor the yoke may be provided.

As noted above, the footwear system may comprise a footwear body and a yoke, and the yoke may be configured to rotate relative to the footwear body in a sagittal plane in dorsiflexion and plantarflexion. In some embodiments, the yoke may also be removably engaged to the footwear body. For example, the yoke may be rotatably connected to the footwear body via a hinge or other suitable fastener, and the yoke may be disconnected to allow for a different yoke having different functional characteristics to be employed by the user. By permitting interchangeability of the yoke, different lower limb stability and propulsive support profiles may be realized for a given article of footwear. In some embodiments, the footwear body may be further engageable with the yoke via eyelets, hooks, or other structures that the laces of the footwear may be engaged with. Thus, the laces may be used to secure the yoke in a desired orientation relative to the footwear body while still permitting dorsiflexion and plantarflexion of the user's foot.

In some embodiments, the yoke may be constructed and arranged to such that the yoke has insufficient stiffness to support restorative forces applied by an associated energy storage device during movement of a user according to embodiments disclosed herein. In some such embodiments, one or more supports may be provided in conjunction with the yoke to provide sufficient stiffness to the yoke to support the forces applied via the energy storage device and permit the yoke of the restorative energy system to store and release energy during a gait cycle of a user wearing the associated footwear. Thus, the lateral stiffness of the yoke may also be selectively altered by adding and removing supports having various stiffness characteristics and profiles.

In instances where an exoskeleton is used, the exoskeleton may be of any suitable size, shape, or other characteristic to promote propulsive support, drop-foot support, lateral support, and/or lower limb stability for a user. As would be appreciated by one of skill in the art, “drop-foot” refers to a condition of weakness that makes it difficult to lift one's toes off the ground, thus resulting in the dragging of one's toes. Such a condition is common after a stroke or the like. Accordingly, the exoskeleton may be constructed and arranged to provide drop-foot support to prevent the foot from dropping by limiting the amount of plantar flexion that a user may experience while the exoskeleton is worn. As noted above, the exoskeleton may include a base and/or a yoke which may be rotatably connected to one another. The base and/or yoke of the exoskeleton may also be positioned between layers of an article of footwear, internal to the footwear, external to the footwear, or a combination thereof as the disclosure is not so limited.

In some embodiments, the base and the yoke may be made of the same material or different materials. In some such embodiments, the base and the yoke may be configured to behave differently from one another under load. For example, the base and the yoke may have different characteristics such as thickness, shape, and durometer values. The base and the yoke may also include different amounts of reinforcement and/or ribbing relative to one another. For example, the yoke may have greater amounts of structural reinforcement and ribbing than the base.

In some embodiments, the base and the yoke may be constructed of different materials. For example, the base and/or yoke may be constructed of a three dimensional printed, molded, or other appropriately formed material such as nylon, thermoplastic polyurethane (TPU), polyurethane (PU), other appropriate polymers, metals, combinations of the forgoing, or other appropriate material. In some instances, the yoke and/or base may be constructed of the same material as the corresponding footwear to which the base and/or the yoke are intended to be attached. Regardless of whether the base and the yoke are constructed from the same or different materials, in some embodiments the exoskeleton may be constructed and arranged to be semi-soft such that when no supports are attached to the exoskeleton, there will be minimal disturbance to the range of motion and activity performance of a user when engaging in the gait cycle. In turn, when supports (e.g., semi-rigid or rigid members) are added to the exoskeleton, it will change the performance of the system and permit varying stiffness profiles for the user. While the exoskeleton may be constructed to be semi-soft, in other embodiments the exoskeleton (e.g., the yoke portion) may be constructed to be semi-rigid or rigid (e.g., a rigid fabric or leather material). As used herein, the terms soft, semi-soft, semi-rigid, and rigid are used to refer to construction arrangements of the exoskeleton and/or the supports having flexure profiles of varying rigidity such that varying levels of compliance and support may be provided to the ankle of a user depending on a desired application.

In some embodiments, the base and/or the yoke may be made using any appropriate manufacturing process or combination of manufacturing process. For example, the base and/or yoke may be manufactured using injection molding, over molding, three dimensional printing, weaving, knitting, or sewing, adhesive bonding, riveting, material-shaping or any other suitable method. While such examples are disclosed above, the yoke and the base may be constructed and arranged using any suitable materials, manufacturing processes, and fabrication processes as the disclosure is not so limited.

The base and yoke may be constructed and arranged to be rotatably connected to one another as disclosed herein. The base and yoke may be connected using any suitable connection means including, but not limited to mechanical hinges such as a pin hinge, an interlocking teeth hinge, rivets, axels, fulcrums, living hinges, or any other suitable hinge type connection or other rotatable connection. In the case of a living hinge, the base and the yoke may be connected to one another via a deformable zone (e.g., an elastomeric zone). As such, in some embodiments, the yoke and the base may overlap with one another. The amount of overlap between the yoke and the base may selectively alter the stiffness of the exoskeleton and the footwear system for a user (e.g., greater overlap between the base and the yoke may increase stiffness in the system). In some embodiments, the supports which may be engaged with the yoke and/or base may also overlap with the other of the yoke and/or base.

As disclosed herein, a footwear system and/or a restorative energy system may include an energy storage device to promote the storing and/or releasing of energy to assist with movement of the user's foot and ankle during various portions of a gait cycle. The inventors have found that during the gait cycle, the peak demand for energy occurs after midstance as the ankle is in the process of increasing dorsiflexion and then rapidly increasingly plantarflexion. In this process, the Achilles tendon, the soleus muscles, the gastrocnemius muscles, as well as other muscles and connective tissues of a user contribute to provide the energy needed during peak demand. In particular, the ankle system may consume 0.2 to 0.5 W/kg of power during dorsiflexion and between 2 to 4 W/kg of power during the transition from dorsiflexion to plantarflexion. Thus, by providing an energy storage device, at least a portion of the desired energy expended during a gait cycle of a user may be offset or supplemented by the energy stored and released by the energy storage device.

In some embodiments, a suitable energy storage device may include, but is not limited to elastic cords, woven cords, springs, elastic bands, bungee cords, elastic sheets, elastic ribs, shaped elastic component (e.g., formed of any suitable shape from injection-molding, compression molding, 3D printing, etc.), die-cut elastic components, elastic-reinforced fabrics or other structural members, or any other suitable energy storage device capable of elastically deforming to store and release energy. For example, a 20 mm×20 mm square patch of elastic-reinforced fabric may be provided and have a spring stiffness of greater than or equal to 500 N/m, 1,000 N/m, 20,000 N/m, or any other suitable spring stiffness as values both greater and lesser than those noted are also contemplated. The energy storage device may also be comprised of suitable elastic or elastomeric materials such as rubbers, silicones, thermoplastics, urethanes, or any other suitable material. The embodiments of energy storage devices disclosed herein may be attached to the footwear body and/or the exoskeleton in any suitable arrangement as the disclosure is not so limited. For example, the energy storage device may be connected between a portion of the footwear body and the yoke, between the base and the yoke, or any other suitable connection arrangement. The energy storage device may also be located externally, internally, and/or integrally formed with a footwear as the disclosure is not so limited.

The one or more supports, or stiffeners as they may also be referred to as herein, may be provided at any suitable location of the footwear as well as the base and/or yoke of the exoskeleton. In some embodiments, a single support may be provided in the footwear and/or exoskeleton. In other embodiments, however, a plurality of supports (e.g., 2, 3, 4, or more) may be provided. In some embodiments, the supports may be provided on either the lateral and/or medial side of the footwear as well as at locations above and/or below the ankle joint of a user when the footwear is worn. This may help to provide varying stiffness profiles for the user. In a preferred embodiment, the inventors have recognized particular benefit with positioning a support on the medial side of the yoke in a footwear system, as the medial side of the yoke may provide the greatest lateral support with an incorporated stiffener by comparison to the other locations of the footwear. Nevertheless, the supports may be provided in any suitable arrangement and number along any suitable location of the footwear and/or exoskeleton as disclosed above.

The supports may also be of any suitable size, shape, material composition, or other characteristic as the disclosure is not so limited. In some embodiments, the supports may also have features formed thereon such as connectors (e.g., detents) for registering a height of the supports, notches for interfacing with a desired pivot location, stiffening ribs, geometries with varying bending and flexure profiles, and other suitable features as described in greater detail below.

The supports may have any suitable dimensions to promote lateral support of the ankle joint of a user while not obstructing plantarflexion and/or dorsiflexion of a user's foot. In some embodiments, the supports may be of a suitable longitudinal (e.g., length) dimension greater than or equal to 20 mm, 30 mm, 40 mm, 50 mm, 60 mm, 70 mm, 80 mm, 90 mm, 100 mm, 110 mm, 120 mm, 130 mm, 140 mm, or greater. The supports may also be of a suitable longitudinal dimension lesser than or equal to 150 mm, 140 mm, 130 mm, 120 mm, 110 mm, 100 mm, or lesser. In some embodiments, the supports may be of a suitable transverse (e.g., width) dimension greater than or equal to 5 mm, 10 mm, 15 mm, 20 mm, 25 mm, 30 mm, 35 mm, 40 mm, 45 mm, 50 mm, 55 mm, or greater. The supports may also be of a suitable transverse dimension lesser than or equal to 60 mm, 55 mm, 50 mm, 45 mm, 40 mm, 35 mm, 30 mm, or lesser. In some embodiments, the supports may be of a suitable thickness greater than or equal to 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, or greater. The supports may also be of a suitable thickness lesser than or equal to 2 mm, 1.9 mm, 1.8 mm, 1.7 mm, 1.6 mm, or lesser. The thickness of the supports could also vary across the longitudinal and/or transverse dimensions of the supports (e.g., one end portion of the supports may have a greater thickness than an opposing end portion to provide a desired stiffness profile). Combinations of the foregoing dimensional parameters for the supports are also contemplated, and values both greater and lesser than the aforementioned values may be used. In a preferred embodiment, the inventors have found particular benefit with providing a support having a longitudinal dimension of between about 80 mm and 120 mm (e.g., 100 mm), a transverse dimension of between about 20 mm and 40 mm (e.g., 30 mm), and a thickness of between about 0.5 mm and 1.5 mm (e.g., 1 mm). With regards to the above dimensions, the various dimensional parameters may refer to a maximum transverse, maximum longitudinal, and/or maximum thickness dimension of the support in some embodiments.

In some embodiments, the supports may be selectively removable from the portion of the article of footwear and/or the exoskeleton to which they are secured or positioned. In some arrangements, upon removal of the one or more supports, the footwear may function similar to footwear where no such supports are provided. The removability of the supports may also permit interchangeability of the supports to yield varying stiffness and flexure profiles as disclosed herein. For example, as a user is progressing through the stages of healing a lower limb injury, select locations of the lower limb may require additional or reduced stiffness. In such an example, two supports may be provided in a footwear system with one support being located at the medial side of the yoke and the other being located at the lateral side of the yoke. At a later stage of healing, the support located at the lateral side of the yoke may no longer be necessary and may thus be removed. Alternatively, the stiffness of the one or more supports provided on either side of the ankle may be changed to be appropriate for the specific stage of healing and/or activity to be performed by a user. While such an example is disclosed, the one or more supports may be provided at any suitable location of the medial and/or lateral side of the footwear body, the base, and/or the yoke at any suitable stage of healing or for any suitable application as the disclosure is not so limited. Further, in some embodiments, a single support located above an ankle joint and positioned on a medial side of the footwear when the footwear is worn may be desirable to provide an appropriate level of support and flexibility of the footwear during use. In some such embodiments, the single support may be spaced at a selectable variable distance relative to a pivot point of the footwear system. For example, the support may be connected to an exoskeleton and/or a footwear at one of a plurality of predetermined locations (e.g., by using appropriately arranged connectors) to change a distance between a distal end portion of the support and the pivot point of the footwear system (e.g., between a closest position relative to the pivot point and a furthest spaced apart position relative to the pivot point). By providing the support at a distance relative to the pivot point, the stiffness of the system may be reduced as compared to implementations where the distal end portion of the support is disposed closer to the pivot point. In some embodiments, the single support may be engaged with the pivot point of the footwear system and overlap the pivot point at a certain distance to provide an increased level of stiffness. While such arrangements are disclosed, a single support may be provided in any suitable arrangement as the disclosure is not so limited.

In contrast to embodiments where the supports are selectively removable as detailed above, in some embodiments the supports may be permanently retained on a corresponding article of footwear and/or the exoskeleton, e.g., the base or the yoke.

In some embodiments, the inventors have recognized that it may be desirable for the supports to be of a desired bending stiffness to provide a certain amount of lateral support or propulsive support to an ankle of a user. In some embodiments, the bending stiffness of a given support may be greater than or equal to 30 Newtons per meter (N/m), 50N/m, 75 N/m, 100 N/m, 300 N/m, 500 N/m, 1,000 N/m, 3,000 N/m, 4,000 N/m, 5,000 N/m, 6,000 N/m, 7,500 N/m or greater. A suitable bending stiffness of a support may also be lesser than or equal to 10,000 N/m, 7,500 N/m, 6,000 N/m, 5,000 N/m, 4,000 N/m, 3,000 N/m, 1,000 N/m, or lesser. The aforementioned bending stiffness values of the supports may be provided in either the sagittal direction or the lateral direction, and values both greater and lesser than the aforementioned values may be used. In some embodiments, the bending stiffness in the sagittal direction may be greater or lesser than the bending stiffness in the lateral direction. In a preferred embodiment, the inventors have found particular benefit with providing a support having a lateral bending stiffness between or equal to 4,000 N/m and 6,000 N/m (e.g., 5,000 N/m).

In some embodiments, the supports may be constructed of a suitable material including, but not limited to plastics, urethanes, metals, composites, fiber composites (e.g., carbon fiber composites), combinations of the forgoing, and/or other suitable materials. The supports may also be constructed of a dynamic material, such as rate-adjusting damper material. As noted above, the supports may also be interchanged with one another. In such an arrangement, supports having materials with varying stiffness profiles may be interchanged with one another to change the amount of lateral and/or propulsive support for a user as needed. For example, when used for healing an ankle sprain, supports with decreasing stiffness may be progressively interchanged for one another (e.g., a semi-rigid support may be changed out for a semi-soft support with a lower stiffness in the lateral and/or sagittal direction when worn by a user) as a user progresses through the healing process.

In some embodiments, the supports may be of a suitable geometry to provide a certain stiffness profile. A suitable two-dimensional geometry of a support as viewed in plan view may include, but is not limited to a rectangular shape, a trapezoidal shape, an oval shape, or any other suitable two-dimensional geometry. In some embodiments, the supports may include appropriate stiffening three-dimensional geometries to provide a certain stiffness profile including, but not limited to a convex shape, a concave shape, a progressively curved shape, and a saddle shape. Thus, it should be noted that the currently disclosed supports are not limited to any particular type of support geometry size and/or shape as the disclosure is not so limited. Without wishing to be bound by theory, in some embodiments, the embodiments of supports disclosed herein may not rely solely upon their intrinsic levels of stiffness, but may also serve to increase the stiffness of the materials around them by altering the shape or the relative position of the surrounding material layers (e.g., footwear outer layer, footwear line, etc.) relative to one another.

In some embodiments, the supports may include one or more notches formed thereon. The notches may be configured to facilitate rotation of a pivot location of the footwear body and/or the exoskeleton (e.g., about the ankle joint of a user during use). Specifically, the notch may be oriented towards a location of the expected pivot location (e.g., the rotation of the ankle joint when worn). In some instances the notch may extend partially around the pivot location when worn while providing an open area of the notch coincident with the pivot location to provide a reduced stiffness at the pivot location. As noted above, the yoke and the base may rotatably connected to one another via any appropriate rotatable connection. Accordingly, in one example, one or more supports may be located on the yoke and/or base, and the supports may include notches that are oriented towards an axis of an associated ankle joint when worn. In another example, one or more supports may be located on a footwear body and/or the yoke of a footwear system, and the supports may be associated with a corresponding pivot point of the footwear body. By orienting a notch of the supports towards the pivot point, a greater degree of stiffness may be provided by the supports (e.g., since the support is closer to the pivot point) while still permitting rotation of the overall footwear.

The one of more notches of the supports may be of any suitable size, shape, or other characteristic as the disclosure is not so limited. In some embodiments, the notch may be of a suitable shape including, but not limited to a semi-circle, semi-ovoid, square, rectangle, or any other suitable shape with an opening formed between two protrusions extending longitudinally away from a main body of the support as the disclosure is not so limited.

While embodiments related to the use of a notch is described above and/or supports spaced apart from a pivot point (e.g., ankle joint of a user) when a footwear is worn are described above, in some embodiments, the pivot point may overlap with the supports. In such an embodiment, the pivot point may be physically connected to the supports via a mechanical connector (e.g., a rivet, a nut, a bolt, a washer, etc.) such that the support at least partially overlaps the pivot connection and is connected or otherwise mechanically engaged to the pivot point. In some embodiments, this mechanical connection may be a rotatable connection between the support and the pivot point. The amount that the support overlaps the pivot connection may also influence the stiffness profile of the support. For example, a support that extends the entire length of a lateral or medial side of an article of footwear may provide greater stiffness and in turn lateral support, but may limit the propulsive support of the user's foot. Said in a different way, the relative distance between the support and the connection point can change the stiffness profile of support.

In contrast to the embodiments detailed above, in some embodiments the supports may not overlap and/or be directly connected to the pivot point of a piece of footwear. Instead, the supports may be positioned at a selectable variable distance away from the pivot point (e.g., at one of a plurality of predetermined positions). Such an arrangement may provide a lesser amount of lateral support than when the supports are directly mechanically engaged to and/or overlap with the pivot connection.

In some embodiments, the supports may include one or more connectors to permit adjustability in the positioning of the supports. In particular, the one or more connectors may be configured to control an amount of a length of a corresponding support that is received in a corresponding receptacle. For example, different portions of a length of a support may be inserted into the receptacle when a support is connected at different locations to the connector including both partial insertions and full insertions into the receptacle. While such arrangements are disclosed, the supports may be retained at any suitable position within the receptacle at any suitable distance from the pivot point to provide a varying stiffness profile of the footwear system as the disclosure is not so limited. Thus, a desired position and orientation of the supports relative to a corresponding pivot point of the footwear body and/or the exoskeleton may be controlled as needed. The connectors may take the form of detents, holes, or other appropriate types of connectors that may allow the supports to be secured at a desired incremental positions on the footwear and/or exoskeleton relative to an associated ankle joint of the user when worn. For example, a given support may have three holes incrementally positioned and corresponding to a low, medium, and high position (e.g., the distance from the respective holes to the pivot point). As will be described in greater detail below, the supports may be received by one or more corresponding receptacles formed on the footwear body and/or the exoskeleton. Accordingly, in some embodiments, the supports may be secured at a desired position by fastening the support directly to the footwear body and/or exoskeleton, or by fastening the footwear through a corresponding receptacle in which the support is disposed when worn by a user. As noted above, the distance at which the support is fastened may influence the amount of lateral and/or propulsive support provided in the footwear system for the user.

In some embodiments, the supports may include corrugations in the form of a corrugated surface extending along a length of one or more surfaces of the supports to alter the directional stiffness of the supports for a given application. The corrugations may be arranged along any suitable location of the supports as the disclosure is not so limited. For example, to address an inversion injury, corrugations may be included in a support that are oriented to provide a greater directional stiffness in the inversion direction and in turn a lesser directional stiffness in the eversion direction. Similarly, to address an eversion injury, corrugations may be included in a support that are oriented to provide a greater directional stiffness in the eversion direction. As such, in some embodiments, the supports may be configured to have a first lateral bending resistance in a first direction and a second lateral bending resistance in a second direction opposite to the first direction, and the first lateral bending resistance may be different than the second lateral bending resistance. While these examples are disclosed, other arrangements of directional stiffness for a given support are possible. For example, in some embodiments, corrugations in a support may be constructed and arranged to provide a greater directional stiffness in a sagittal direction to promote propulsive support.

In some embodiments, the supports may include one or more stiffening ribs formed thereon and extending along at least a portion of a length or other appropriate dimension of the support. The one or more stiffening ribs may be of any suitable size, shape, number, or other characteristic as the disclosure is not so limited. In some embodiments, a suitable number of stiffening ribs may be greater than or equal to 1, 2, 3, 4 or more stiffening ribs. In other embodiments, however, no stiffening ribs may be provided on a given support. The stiffening ribs may also be selectively positioned on an associated support to provide directional stiffness to a select region of the support. For example, in some embodiments, a stiffening rib may be oriented to provide greater directional stiffness in an eversion direction, an inversion direction, and/or in a sagittal direction.

In reference to the embodiments of the supports disclosed herein, the user may selectively alter the position and/or orientation of the supports to yield different stiffness characteristics. In some embodiments, the supports may be rotated, flipped, moved further away from or closer to the pivot point, exchanged with supports with different stiffness, or other appropriate modifications to yield a different stiffness profile. For example, if a support has a flexure profile that is biased towards the eversion direction, the support may be flipped to provide bias towards the inversion direction.

The footwear and/or exoskeleton may include one or more receptacles which may be configured to receive the one or more supports as referenced above in any suitable fashion. The one or more receptacles may be sealed or otherwise enclosed to prevent the supports from being easily removed from the receptacles. In other embodiments, however, the receptacles may be configured such that the receptacles may be opened, or have a permanently exposed opening, to permit removability and interchangeability of the supports.

In some embodiments, the receptacles may take the form of a variety of suitable engagement features to permit adjustability of the positioning of the supports on the footwear and/or the exoskeleton to which the supports may be connected. In some embodiments a suitable receptacle may include, but is not limited to slots, pockets, loops, connections (e.g., mechanically interlocking features, snape connectors, threaded fasteners, etc.), or any other suitable type of receptacle configured to receive one or more supports as the disclosure is not so limited. For example, pockets may be provided by sewing material into a layer of the footwear body (e.g., the liner and/or outer layer). In such an example, the shape and/or size of the receptacle may be selected to help secure the corresponding support to prevent inadvertent adjustment of the support in the anterior or posterior directions. In another example, slots may be formed in the base and/or yoke of the exoskeleton, and the supports may be configured to engage with the slots at a certain incremental position. While such examples are disclosed, the supports may be fastened to the footwear body and/or exoskeleton using any suitable type of method as the disclosure is not so limited.

In some embodiments, the receptacles may be positioned along any suitable side (e.g.,, lateral or medial sides) of the footwear body and/or the base and yoke of the exoskeleton as the disclosure is not so limited. In some embodiments, the receptacles may be positioned above and/or below a pivot point of the footwear and/or the exoskeleton. In other embodiments, however, the receptacles may be positioned away from the pivot point to provide a lesser amount of support for a given application.

The footwear body itself may be constructed and arranged to include a variety of different layers and other features for a given application. In some embodiments, the footwear body may include an outer layer, an optionally provided liner material layer, and an optionally provided cushion layer. The outer layer may be constructed of a variety of suitable materials including, but not limited to leather, canvas, synthetic materials, or any other suitable material as the disclosure is not so limited. A liner material layer may be included and arranged internally to the outer layer. The liner material layer may include any suitable material such as a soft fabric or leather, and may be optionally waterproof. The cushion layer, if included, may be provided between the liner layer and the outer layer to provide an increased amount of padding and comfort for the end user. While the above arrangement is disclosed, the footwear may be constructed and arranged in any suitable configuration as the disclosure is not so limited, and the embodiments disclosed herein may be applied for use in host footwear of any suitable type.

In some embodiments, the inventors have recognized that the shape and size of the footwear materials itself may influence the stiffness of the overall footwear system. For example, the contour of the footwear material that is proximal to one or more supports which may be included in the system may be formed to selectively increase or decrease stiffness. In particular, a more rigid outer layer may increase stiffness and thus prevent buckling. The orientation of the outer and inner layers (e.g., liner layer and cushion layer) may also yield a varying amount of stiffness in the footwear system. For example, one or more supports may be positioned in the system such that the volume between two or more layers of the footwear is altered to increase the stiffness of the footwear itself, an exoskeleton yoke and/or an exoskeleton base.

The inventors have recognized that in some cases, the lower limb of a user may experience a sufficiently high amplitude landing force in a non-vertical direction (e.g., due to uneven ground) which may result in injuries resulting from rotation of the ankle (e.g., ankle sprain). While strapping or taping of the ankle can protect the ankle, this may transmit torque from a landing force to other body parts of the user (e.g., leg, knee, hip, etc.).

In view of the above, the inventors have recognized that benefits may be realized by providing a footwear system with a supplemental shock absorber disposed between the footwear and the ground. This may allow the mechanism to twist, deform, and absorb energy so that the body of a user does not have to manage the full load. In order to achieve these features, a fulcrum may be established between the heel of an article of footwear and an added outsole heel of the footwear. The fulcrum may include a shock absorber for shock attenuation on either side which may be configured to absorb and dissipate at least a portion of the applied shock and associated energy. The inventors have found that the use of a shock absorber configured to dissipate energy may serve to dampen a high amplitude landing force that may be experienced by a user (e.g., due to parachuting or other high impact activities). The shock absorber may correspond to any appropriate compressible structure capable of absorbing and dissipating energy include crushable pucks of dampening material (e.g., foam, sponge, crushable matrix), 3D printed collapsible lattice, airbags, variable shear dampers (e.g., deformable dampening straps), or any other suitable shock absorber. The above described shock absorber arrangements may serve to limit injuries associated with the lower limb of a user by providing added stability and dampening properties.

In some embodiments, a footwear system may include one or more lateral dampers to provide support against inversion and/or eversion of a user's ankle. The lateral dampers may include variable viscosity dampers or other friction dampers and rip straps or other suitable energy dissipating arrangements. These dampers may be secured to the footwear using any suitable arrangement including, for example, through stirrups which may affix the strap across the bottom of the heel to connect the strap between the medial and lateral sides of the footwear. The straps or other suitable dampers may then engage with one or more anchor points or receptacles according to embodiments disclosed herein. Regardless of the orientation of the dampers, by traversing the dampers between portions of the footwear system, a larger radius and pathway for energy dissipation may be realized, thus helping to limit and/or prevent inversion and/or eversion injuries.

In some embodiments, the embodiments of a restorative energy system disclosed herein may be provided as a kit to a manufacturer or other user independently from an article of footwear to which the system is intended to be engaged. Such an arrangement may provide benefits as it would enable a footwear manufacturer without expertise in exoskeleton fabrication to incorporate the aforementioned kit into a given article of footwear without having to also fabricate the exoskeleton.

Such a kit referenced herein may include, but is not limited to a footwear liner material, a base of an exoskeleton, a yoke of an exoskeleton, a rotatable connection between the base and the yoke, and an energy storage device, among other components. In some embodiments, the footwear liner may be pre-sewn to match at least a portion of the internal shape of a host footwear to which the kit is intended to be employed. The base and the yoke may each individually, or in combination with one or more supports, may exhibit sufficient stiffness to prevent buckling during use. The rotatable connection provided between the base and the yoke to permit the yoke to be rotatable relative to the base may include a mechanical hinge, a deformable zone, or any other suitable type of rotatable connection as the disclosure is not so limited. The rotatable connection may also help prevent buckling under load and may help to preserve stability by providing rigidity near the ankle joint in the plantarflexion and dorsiflexion axis of rotation in some embodiments. The energy storage device may be of any suitable type as disclosed herein. The footwear liner, other portion of the footwear, and/or an exoskeleton may also include one or more receptacles configured to retain one or more supports and maintain the one or more supports in a desired orientation relative to the footwear during use according to embodiments disclosed herein.

The kit described above may be formed in a series of method steps. For example, the kit may be entirely pre-fabricated and prepared for use in inserting into a corresponding host footwear, or the kit may be provided as a plurality of parts intended to be fabricated by a manufacturer or other user for insertion into the host footwear. Such arrangements may reduce the amount of design and manufacturing needed by a manufacturer to incorporate the footwear system into a select article of footwear, thus improving manufacturing efficiency.

As detailed above, a variety of suitable embodiments may be employed to control the amount of lateral and/or propulsive support provided in a footwear system and/or restorative energy system. In some embodiments, the footwear, supports, and/or the restorative energy systems may provide a combined stiffness for the overall footwear system. Again, the overall combined stiffness may be selected to provide a desired amount stiffness for a desired application and/or different stages of injury healing. For example, appropriate stiffness ranges may include the following general high, medium, and low levels of support for different applications of 6 to 12 Nm of resistance at 30 degrees of deflection in the lateral direction, 3 to 6 Nm of resistance at 30 degrees of deflection in the lateral direction, and 0 to 3 Nm of resistance at 30 degrees of deflection at 30 degrees of deflection in the lateral direction, respectively. Combinations of these ranges may be used to provide a desired amount of lateral support depending on the embodiment. Thus, the inventors have recognized that, in a preferred embodiment, the footwear system of embodiments disclosed herein may permit interchangeability and/or other adjustments of the supports to provide the support ranges noted above for the “high”, “medium”, and “low levels”. While these ranges of support are disclosed, in some embodiments, the footwear system disclosed herein may provide no additional support as compared to a typical footwear. By providing such ranges of lateral support, the device will allow for the user to progressively heal from an associated lower limb injury (e.g., an ankle sprain) or allow the user to prevent the injury altogether while only using a single system.

While a variety of suitable articles of footwear have been referenced above, the embodiments disclosed herein may be employed for use in any suitable article of footwear including, but not limited to boots (e.g., hiking boots, work boots, tactical boots), high-top athletic sneakers, high-top shoes for the medical industry (e.g., doctors, nurses, waitstaff), dress shoes, low top sneakers, slippers, or any other suitable footwear type. It should be understood that in instances where lower profile footwear is used that may not extend above the ankle, appropriate structural features, such as a yoke, or other portion of the disclosed systems and devices may extend above an opening of the footwear to engage with an appropriate portion of the lower leg (e.g., ankle or calf) as the disclosure is not limited to only being used in boots.

The inventors have realized that the embodiments disclosed herein may provide a variety of benefits to an end user when provided for use in a footwear system and/or a restorative energy system. Such benefits include a wide array of adjustability of lateral and/or propulsive support for the ankle of a user, reduced occurrence of lower limb injuries, the ability to promote recovery of an injury of a user by adjusting and interchanging supports, providing targeted support and/or flexure by altering the individual characteristics of supports, and many other benefits. The inventors have also realized that benefits may be realized by reducing the level of support in a footwear system according to embodiments disclosed herein (e.g., such that there is no additional support) to a level comparable with traditional footwear. In such an embodiment, the one or more supports disclosed herein may be disengaged from the footwear system to optimize agility and comfort of the user when the footwear system is worn by the user while also allowing the user to engage one or more supports when needed for a certain terrain, activity, an incurred injury, or other suitable application.

The embodiments of footwear systems and restorative energy systems having supports disclosed herein may be used in any suitable application where it is beneficial to have an adjustable level of support in an article of footwear. For example, potential applications of the embodiments disclosed herein including post-injury recovery, post-operative recovery, and/or injury prevention in fields where having controllable support in footwear is important such as hiking, sports, the workplace, policing, the medical industry, or any other suitable field as the disclosure is not so limited. Moreover, commonly used terms disclosed herein are described by the following.

The term “yoke” is used herein to describe a portion of an exoskeleton engageable with an upper portion of a footwear body, where the yoke is intended to be located near a portion of a user's lower limb, which may also be referred to as a leg, at or above the user's ankle joint.

The term “base” is used herein to describe a portion of an exoskeleton engageable with a lower portion of a footwear body, where the base is intended to be located near a portion of a user's lower limb at or below the user's ankle joint.

The term “sagittal plane”, also known as the “longitudinal plane”, is used herein to define a vertical plane which spans from the front to the back of the user such that the plane anatomically defines right and left halves of a user's body. This term is used herein in reference to the direction of propulsive movement of a user's body during a gait cycle where the general direction of movement of a user during forward movement (e.g., walking, running, etc.) may be oriented in a horizontal direction parallel to the ground and the sagittal plane when the footwear is disposed on a supporting underlying surface.

The term “frontal plane” is used herein to define a vertical plane which spans from the left to ride side of the user such that the plane anatomically defines front and back halves of a user's body. This term is used herein in reference to the direction of lateral support of a user's body during the gait cycle wherein the lateral direction may be taken in a horizontal direction parallel to the ground and the frontal plane when the footwear is disposed on a supporting underlying surface.

The term “lateral side of footwear” is used herein to describe a portion of the footwear that is oriented to the outside of a foot of a user. In particular, the lateral side may be further defined as the side of the footwear that is closer to the digitus minimus pedis (i.e., the little toe) of a user's foot.

The term “medial side of footwear” is used herein to describe a portion of the footwear that is oriented to the inside of a foot of a user. In particular, the medial side may be further defined as the side of the footwear that is closer to the hallux (i.e., the big toe) of a user's foot.

The terms “supports” or “stiffeners” are used interchangeably herein to describe the structures that may be provided in a footwear system or a restorative energy system to enhance lateral (e.g., transverse) support to an ankle of a user.

The term “propulsive support” is used herein to describe the promotion of dorsiflexion and plantarflexion in the sagittal plane of a user's foot during a gait cycle.

For the sake of clarity the embodiments described in the figures refer to a boot. However, it should be understood that these embodiments may be applied to any appropriate type of footwear and the reference to boots may be taken to also refer generally to other types of footwear as well as the disclosure is not so limited.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

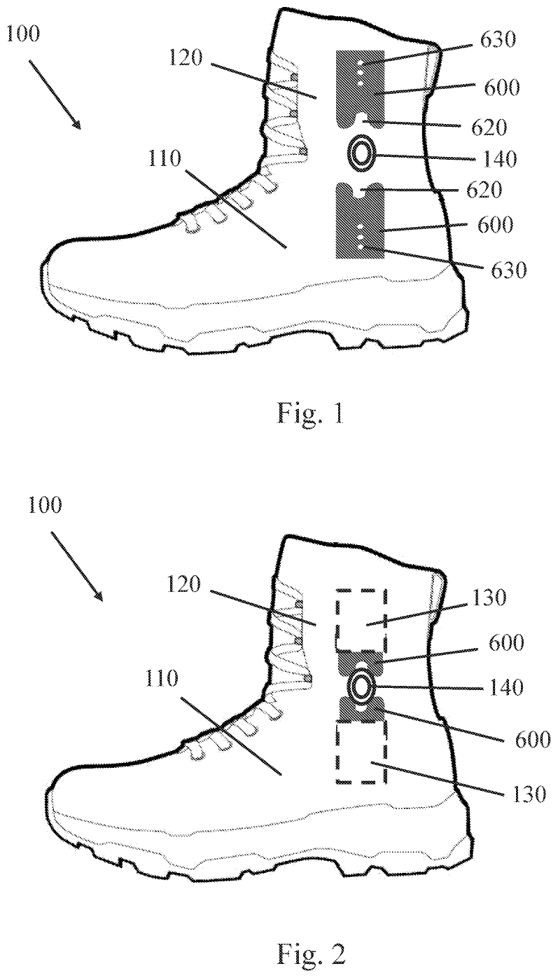

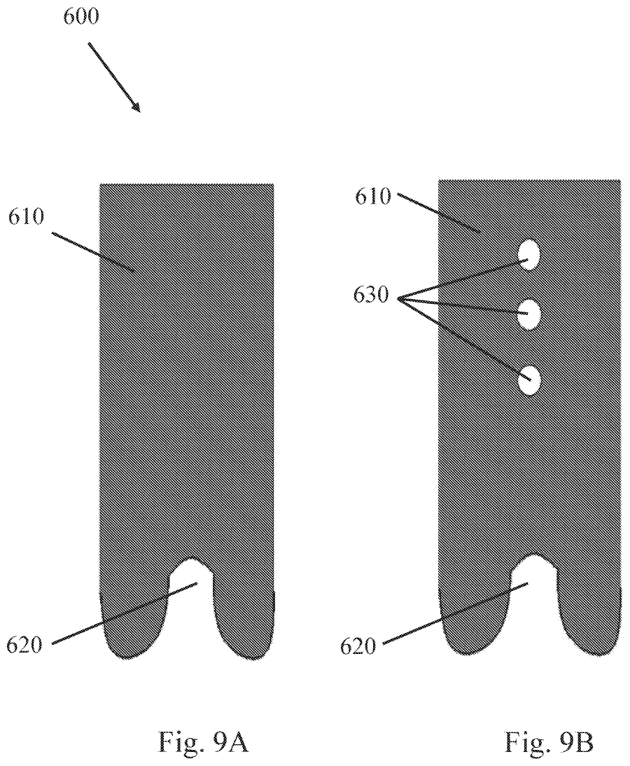

FIG. 1 presents a schematic view of one embodiment of a footwear system 100 having a lower boot portion 110 and an upper boot portion 120. The footwear system 100 may include two supports 600, where one support is associated with the lower boot portion 110 and where the other support is associated with the upper boot portion 120. In the embodiment of FIG. 1, the supports 600 are connected directly to the body of the footwear (lower portion 110 and upper portion 120) using any appropriate type of connection. In some embodiments, the supports 600 may include a notch 620 and/or a plurality of detents 630 (e.g., holes) to permit adjustability in positioning the support, as is described in greater detail in reference to FIGS. 9A and 9B below. The footwear system 100 also includes a pivot point 140 which is located proximal on the footwear to an expected axis of rotation of an ankle joint of a user when the boot is worn. In the various embodiments disclosed herein, a longitudinal axis of a support (e.g., an axis parallel to a largest dimension of the support) may be oriented in a direction that is at least partially directed towards, and in some instances is directed towards, the pivot point of the footwear system. In some instances the longitudinal axis of the one or more supports may be oriented in a direction that is approximately parallel to a vertical direction of the footwear relative to a supporting surface the footwear system when the footwear system is disposed on the supporting surface in a normal orientation during use with the sole of the footwear system in contact with the supporting surface.

In the embodiment of the FIG. 1, the notches 620 of the one or more supports 600 are spaced apart from and oriented towards the pivot point 140 of the footwear from both above and below the pivot point. This separation distance between the supports and the pivot point may affect the overall lateral stiffness of the footwear in the lateral direction around the pivot point. While not shown in FIG. 1, the notch 120 may be positioned such that the notch at least partially overlaps with the pivot point 140 with the associated protrusions extending outwards longitudinally from the support on either side of the notch may be positioned on opposing sides of a rotational axis extending through the pivot point. Alternatively or additionally, the portions of the supports proximal to the pivot point may be connected to the pivot point 140, e.g., via a suitable mechanical fastener as disclosed herein, as shown in FIG. 2.

FIG. 2 presents a schematic view similar to that of FIG. 1, where the footwear system 100 further includes receptacles 130 positioned on the lower boot portion 110 and the upper boot portion 120. As shown in FIG. 2, the receptacles 130 are configured to receive and retain the supports 600 in a desired position and orientation relative to the footwear during use. Additionally, the receptacles may be configured to permit interchangeability of the supports 600 via releasable connections, removal of the support from an opening of the receptacle, detents, combinations of the forgoing and/or any other appropriate construction capable of selectively retaining an associated support in a desired position and orientation. FIG. 2 also shows the notches of the supports 600 as at least partially overlapping with the pivot point 140 to promote lateral support in the footwear system 100 while still providing sufficient flexibility about the pivot point to provide a desired level of propulsive support (plantarflexion and dorsiflexion). In some embodiments, the receptacles 130 may be affixed to or formed in the body of the footwear using any suitable method disclosed herein.

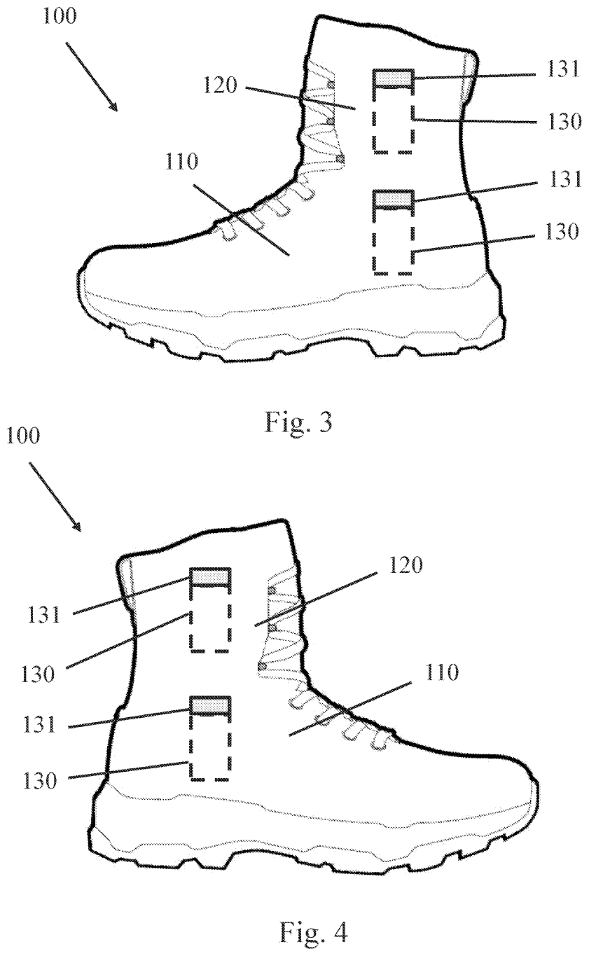

FIGS. 3 and 4 present schematic views of a medial side and a lateral side of a footwear system 100 having a plurality of receptacles 100, respectively. The footwear of FIGS. 3 and 4 may either correspond to the same piece of footwear with supports located on both the lateral and medial sides or separate footwear with supports only on one of the lateral and medial side as the disclosure is not limited in this fashion. In either illustrative embodiment, the receptacles 130 may be positioned at any suitable location along the footwear body (lower portion 110 and/or upper portion 120). This may include receptacles configured to position and maintain an orientation of the associated supports above and/or below the pivot point of the ankle when the footwear is worn. The receptacles 130 may be configured as a pocket, as shown in FIGS. 3 and 4, where the receptacles 130 may be configured to receive corresponding supports to provide increased and/or decreased lateral stiffness in the footwear system 100. However, other constructions for the receptacles may also be used. The receptacles 130 may be openable via an opening 131 to permit interchangeability of the corresponding supports, and thus may enable the use of supports having varying stiffness profiles. In view of the above, the receptacles and supports may be positioned along the lateral and/or medial sides of the footwear body. As disclosed herein and will be described in greater detail below, the receptacles and supports may also be positioned along the lateral and/or medial sides of an exoskeleton (e.g., base and/or yoke).

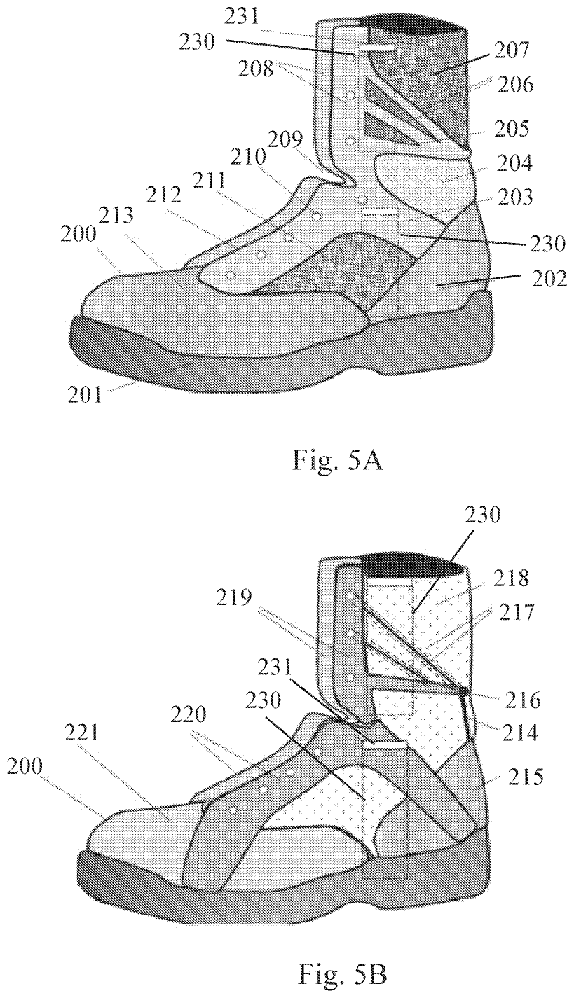

FIG. 5A presents a schematic view of a footwear system 100 showing an outer layer of the footwear while FIG. 5B presents the view of FIG. 5A with the outer layer removed for clarity to show the inner layers of the footwear system 100. In some embodiments, the footwear body may include a plurality of layers such as an outer layer, a footwear body liner, and a cushion layer which may be disposed between the outer layer and the liner to provide increased comfortability for the user. Moreover, the footwear body may be divided substantially into two portions as follows. The first portion is a footwear upper, which may be denoted by the region of the footwear body at or above the location of an ankle joint of the user when the footwear is worn. The second region is a footwear lower, which may be denoted by the region of the footwear body at or below the ankle joint of user when the footwear is worn. The footwear upper and the footwear lower may be configured to receive a base and a yoke of an exoskeleton, respectively.

In addition, as disclosed herein, an energy storage device may be included to provide added tension to the footwear system. In some such embodiments, the energy storage device may be attached to any suitable location along the footwear body (e.g., the footwear upper and/or the footwear lower) as the disclosure is not so limited. The energy storage device may also be attached to the base and/or the yoke of an exoskeleton in embodiments where an exoskeleton is included in the footwear system.

In FIG. 5A, the footwear system includes an outsole 201, a lower collar 203, an ankle collar 207, a quarter panel 211, a toe box 213, and an interior gusset 209 that interrupts a lower eyestay 212 from an upper eyestay 208. The upper eyestay 208 may be sufficiently rigid to support a collar cantilever 205 with the support of at least one cantilever support 206. The cantilever support 206 acts in tension to help connect the collar cantilever 205 with the upper eyestay 208. The eyelets 210 may be used to anchor the cantilever supports 206 to receive further support under tension. As can be more easily seen in FIG. 5B, the upper eyestay 208 and the lower eyestay 212 may be reinforced by upper eyestay reinforcement 219 and lower eyestay reinforcement 220, respectively. The collar cantilever 205 may also be reinforced with the collar reinforcement 216. FIG. 5B also shows a sock liner and padding system 218 which may be at least partially elastic to provide the ability to stretch during movement in the gait cycle. The collar cantilever 205 may be connected to suitable energy storage devices, such as elastic sheet material 204 which may be anchored below the collar cantilever 206 and above the foot collar 203 and heel counter panel 202. A separate energy storage device 214 (e.g., an elastic cord) may also be provided in the system and engaged with the collar cantilever 205 via any suitable mechanical fastener. The internal layers shown in FIG. 5B may also include a structural toe protector 221.

Notably, the embodiments of FIGS. 5A and 5B also include one or more receptacles 230 configured to receive corresponding one or more supports as described elsewhere herein. The receptacles are depicted as pockets including openable ends 231 which may be configured to removably receive the corresponding supports to control the stiffness of the footwear system. As shown in FIG. 5A, the receptacles 230 may be positioned on the outer layer of the boot 200. In contrast, in FIG. 5B, the receptacles 230 are shown as being positioned on the inner layers (e.g., the liner). Moreover, the receptacles 230 and corresponding supports may be positioned on any suitable location of the respective layers. For example, the receptacles 230 may be positioned in the upper region of the boot 200 on the ankle collar 207 or on an upper region of padding 218 while other receptacles 230 may be positioned on the quarter panel 211 or the lower region of padding 218. Again the receptacles may be arranged to position and orient the supports above and/or below the ankle when worn in an arrangement that may either be overlapping with the pivot point of the ankle or spaced apart from the pivot point. While such examples are disclosed, the receptacles and associated supports may be positioned on any suitable layer and at any suitable position and/or orientation on the lateral and/or medial side of the boot.

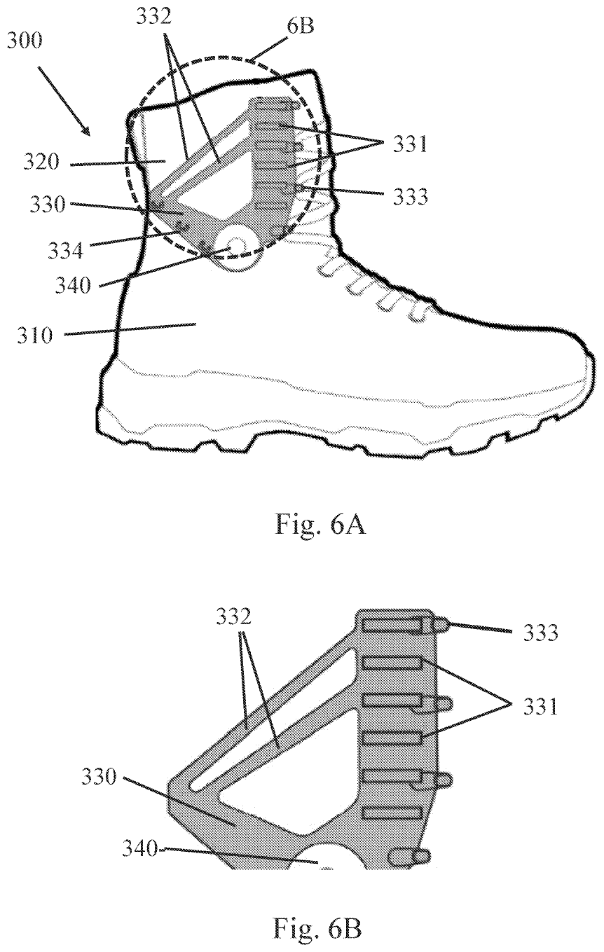

FIG. 6A presents a schematic view of a footwear system 300 having a footwear body with a lower portion 310 and an upper portion 320, where a yoke 330 is engaged with the upper portion 320. The yoke 330 is mechanically fastened to a pivot point 340 such that the yoke 330 may be rotatable relative to at least a portion of the footwear body. The yoke 330 may include receptacles 331 in the form of slots formed thereon, where the receptacles 331 may be configured to receive and lock a corresponding support in a desired position and orientation. However, other types of receptacles may also be used as disclosed herein. The yoke 330 also includes yoke supporting structures 332 to increase rigidity of the yoke 330 and eyelets 333 which are configured to engage with corresponding laces of the footwear to secure the yoke 330 in a desired orientation. The yoke 330 also includes anchor points 334 which may be configured to be engaged with an energy storage device (e.g., an elastic cord) to provide tension and propulsive support to the yoke 330. In some embodiments, as detailed above, the stiffness of the yoke 330 may be insufficient in a lateral and/or sagittal direction to support a force applied to the yoke 330 by the energy storage device during use. In some such embodiments, one or more supports may be provided and engaged with the receptacles 331 of the yoke such that the combined stiffness of the yoke 330 and one or more supports is sufficient to support the force applied to the yoke during use.

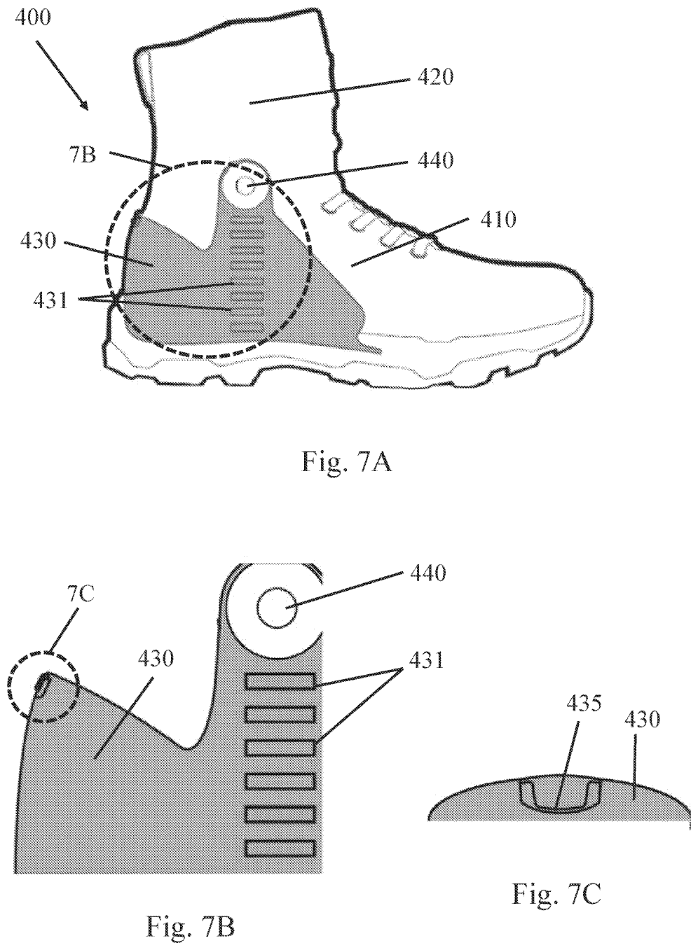

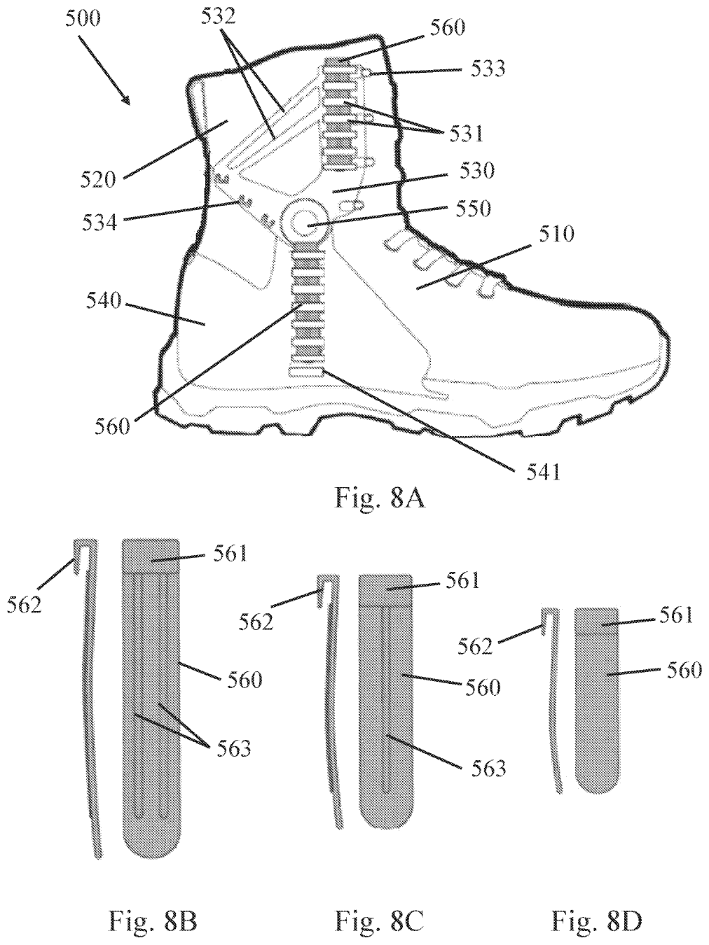

In some embodiments, the yoke 330 may have at least a portion of the yoke 330 that extends at least partially around a perimeter of the upper portion 320 such that the portion extending partially around the perimeter of the footwear may be secured against an opposing side of the upper portion 320 and/or the laces of the footwear body. However, instances in which the yoke is only present on a single side of the footwear are also envisioned. Additionally, in some embodiments, an additional yoke 330 may be positioned on the opposing side of the upper portion 320 to provide increased support to the ankle of the user (e.g., a yoke 330 may be provided on both a lateral and medial side of the upper portion 320 of the footwear body). In some embodiments, the yoke 330 may be configured to be provided on a footwear body in conjunction with a base such as, for example, base 430 shown in FIG. 7A. Additionally, such an embodiment where both a yoke and a base are provided is detailed in reference to FIG. 8A below. The base and the yoke of such embodiments may be rotatably connected to one another at a pivot point (e.g., a rotatable connection positioned at or near the location of a user's ankle when the footwear is worn). FIG. 6B presents an enlarged view of region 3B of FIG. 6A. As can be more easily seen in FIG. 6B, the slots of the receptacle 331 may be positioned vertically along the yoke to permit a support positioned therein to be received and supported on the yoke 330 at a desired position.

FIG. 7A presents a schematic view of a footwear system 400 having a footwear body with a lower portion 410 and an upper portion 420, where a base 430 is engaged with the lower portion 410. The base 430 may be mechanically fastened to a pivot point 440 of the footwear. The base 430 also includes a receptacle 431, for example, in the form of slots formed thereon, configured to receive and lock a corresponding support at a desired position and/or orientation. FIG. 7B presents an enlarged view of region 7B of FIG. 7A, where the slots of the receptacle 431 and the pivot point 440 to which the base 430 is fastened can be more easily seen. FIG. 7C presents an enlarged side view of region 7C of FIG. 7B, where a loop 435 is shown on the base 430. The loop 430 is configured to be attached to an energy storage device (e.g., an elastic band) to provide increased tension to the base 430 and to assist in providing propulsive support to the user. In some embodiments, such as those detailed in reference to FIG. 8A below, the base 430 may be provided in conjunction with a yoke on the footwear body. In some such embodiments, the base may be provided on a lower portion of the footwear body while the yoke is provided on or otherwise connected to an upper portion of the footwear body. The base and the yoke may also be provided on either the lateral and/or medial side of the footwear body as the disclosure is not so limited. The base and the yoke may be connected via any suitable rotatable connection to permit the yoke to rotate relative to the base to both permit dorsiflexion and plantarflexion of the ankle joint and provide propulsive support for a user in one or more portions of the gait cycle. In some embodiments, one of the yoke or the base may overlap with the other of the yoke and the base to provide increased rigidity in the exoskeleton.

FIG. 8A presents a schematic view of a footwear system 500 having both a base 540 and a yoke 530 positioned on a lower portion 510 and an upper portion 520 of a footwear body, respectively. In FIG. 8A, the yoke 530 and the base 540 may be similar to the yoke and base described previously above. Additionally, the yoke and base may be rotatably connected to one another and a pivot point 550 of the footwear to which at least a portion of both the yoke 530 and the base 540 are fastened. Thus, the yoke 530 may be rotated relative to the base 540 and the lower portion 510 of the footwear body. FIG. 8A also shows supports 560 attached to both the yoke 530 and the base 540 via receptacles 531 and 541, respectively. The receptacles 531 and 541 may be configured as any suitable engagement features including, for example, the depicted slots or loops shown in FIG. 8A. On the base 540, the support 560 is disposed in the receptacle 541. However, the external fixation of a support by a receptacle (e.g., via hooks formed on the support engaging with slots, connections, or other external connections) may also be used. A similar arrangement may be present between the yoke 530 and the corresponding support 560. The respective supports 560 located on the base 540 and yoke 530 may be positioned at any suitable position relative to the pivot point approximately associated with an axis of rotation of the ankle joint when worn as the disclosure is not so limited. For example, the support 560 located on the base 540 is located closer to the pivot point 550 than the support 560 located on the yoke 530, and as such, the support 560 on the base 540 may provide greater stiffness to the footwear.

FIG. 8A also shows that the yoke 530 includes yoke supporting structures 532 to increase rigidity of the yoke 530 and eyelets 533 which are configured to engage with corresponding laces of the footwear to secure the yoke 530 in a desired orientation. The yoke supporting structure 532 may include one or more braces that extend between separate portions of the yoke 530 to limit the displacement of these structures away from each other and/or to increase a rigidity of the yoke. For example, the one or more supporting structures 532 may corresponding to the braces extending between a front portion of the yoke with a support disposed thereon and a rear portion of the yoke which may be attached to an energy storage device for applying a desired torque about the ankle joint during one or more portions of a gait cycle. This arrangement may help to prevent or at least limit the displacement relative to one another of these separate portions of the yoke when a restorative force is applied to the yoke during use. This may enable the yoke 530 to provide sufficient propulsive support for the user during the gait cycle. In particular, the braces may enable the yoke 530 to withstand the tension applied to one or more portions of the yoke during dorsiflexion and/or plantarflexion of a user's foot during a gait cycle. The yoke 530 also includes anchor points 534 which may be configured to be engaged with an energy storage device (e.g., an elastic cord) to provide tension and stability to the yoke 530.

FIGS. 8B-D present various embodiments of a support 560 that may be implemented for use in the footwear system of FIG. 8A and/or other footwear system embodiments described herein. In each of FIGS. 8B, 8C, and 8D, the support 560 is shown to include a proximal portion 561 with a hook structure 562 which is configured to engage with a corresponding feature of the receptacle (e.g., loops, openings, slots, etc.) and a body that extends away from the proximal portion 561 towards an opposing distal end portion of the support. However, the sizing of the different supports 560 shown in the different figures is depicted as becoming progressively smaller in each of FIGS. 8B, 8C, and 8D. This corresponds to different size supports providing different amounts of stiffness that may be inserted into and removed from the corresponding one or more receptacles of the footwear system. The different length supports may also result in the supports being spaced further away from or closer to the pivot point which may also further change the resulting stiffness of the footwear. Moreover, as illustrated in FIGS. 8B and 8C, a support may include one or more stiffening ribs extending along at least a portion of a length of a support to increase a stiffness of the support. For example, FIG. 8B includes two stiffening ribs 563 formed thereon, while FIG. 8C includes one stiffening rib 563 formed thereon and FIG. 8D includes no stiffening ribs. Depending on their size, orientation, and position, the inclusion of stiffening ribs may serve to provide a varied stiffness profile to the support as needed. While such arrangements are disclosed above, the supports may be constructed and arranged in any suitable fashion as the disclosure is not so limited.

FIGS. 9A and 9B present further embodiments of supports 600. In FIG. 9A, the supports 600 include a support body 610 having a distal end and a proximal end, where a notch 620 is shown to be formed in the distal end of the support body 610. The notch 620 may consist of an opening formed between two protrusions extending longitudinally away from the support body 610. Moreover, the notch 620 may be oriented towards a location of the expected pivot location (e.g., the rotation axis of the ankle joint) when worn. The notch 620 may be configured to extend at least partially around and be coextensive with a corresponding pivot point of a footwear body and/or exoskeleton to permit lateral support and sufficient compliance relative to the rotational axis of the pivot point to facilitate dorsiflexion and plantarflexion of a user's foot while engaged with the footwear. FIG. 9B shows a similar embodiment of a support 600 to that of FIG. 9A, but includes detents 630 in the form of holes formed on the support body 610. The detents 630 may be incrementally positioned along a longitudinal dimension (e.g., a length) of the support body 610 to allow the support 600 to be secured at a desired position on a corresponding footwear body and/or exoskeleton using corresponding detents, or other types of connectors, formed on the footwear body and/or an associated exoskeleton. For example, a fastener may be engaged with either the upper, middle, or lower detent 630 on the support 600 to secure the support 600 at a corresponding upper middle, or lower position on the footwear and/or exoskeleton which may again adjust the position of the support relative to the pivot point of the footwear.

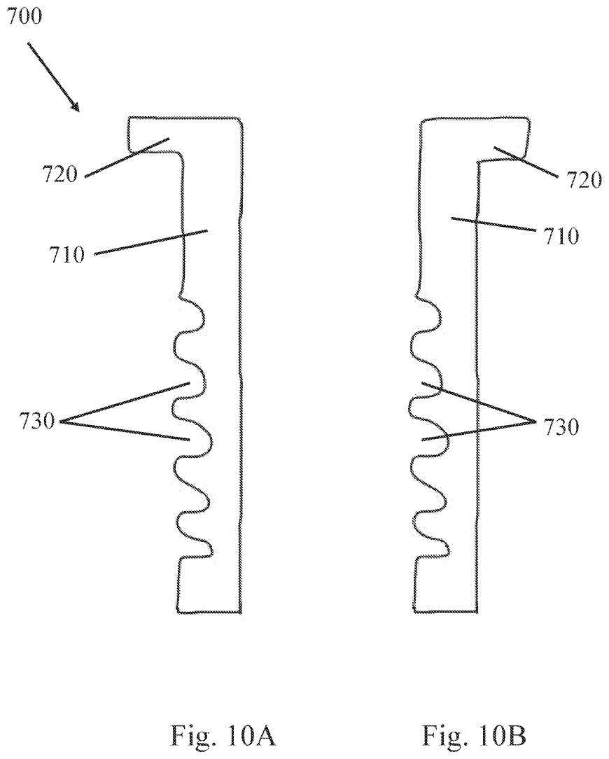

FIGS. 10A and 10B present further embodiments of supports 700 having a targeted bending profile. In particular, in these embodiments, a targeted bending profile is achieved by providing a corrugated surface having a plurality of corrugations 730 that extend along at least a portion of a length of a first surface of a support body 710 of a support 700. The corrugations 730 may be of any suitable number, shape, spacing relative to one another, size, or any other suitable characteristic as the disclosure is not so limit. For example, as shown in FIGS. 10A and 10B, five corrugations 730 may be included in the support 700 that exhibit a smooth curved surface extending between the separate corrugations. However, embodiments in which different geometries including triangular, rectangular, and other appropriately shaped corrugations may be used as the disclosure is not so limited. In some embodiments, the corrugations 730 may extend an entire length of the support 700. In other embodiments, however, the corrugations 730 may only extend along a portion of a length of the support 700. Such variations in corrugation spacing, number, shape, and/or size may result in varying degrees of targeted flexure in the support 700.

A distal end portion of the support body 710 may also include an optional engagement feature 720 (e.g., a hook) to permit engagement of the support 700 with a corresponding receptacle. The supports 700 may have a predefined orientation when engaged with a corresponding receptacle. For example, in some embodiments, the supports 700 may be oriented such that the corrugations 730 may provide targeted flexure of the support in an inversion direction, an eversion direction, and/or a direction in the sagittal plane. Accordingly, by providing various arrangements of corrugations 730 on supports 700, the supports 700 may exhibit targeted flexure profiles that enable support in a desired direction (e.g., the direction of support is opposite the direction of the targeted flexure where corrugations are situated). FIGS. 10A and 10B show supports 700 having opposing flexure profiles. For example, when engagement features 720 are engaged to corresponding receptacles on a medial side of footwear, the support of FIG. 10A may have a greater directional stiffness in an eversion direction while the support of FIG. 10B may have a greater directional stiffness in the inversion direction. While these specific examples are disclosed, the supports 700 may have any suitable number, size, or shape of corrugations 730 disposed along a dimension of the supports 700 to provide targeted flexure. Thus, the depicted supports may exhibit different bending stiffnesses in the inversion and eversion direction when used with the various footwear systems disclosed herein.

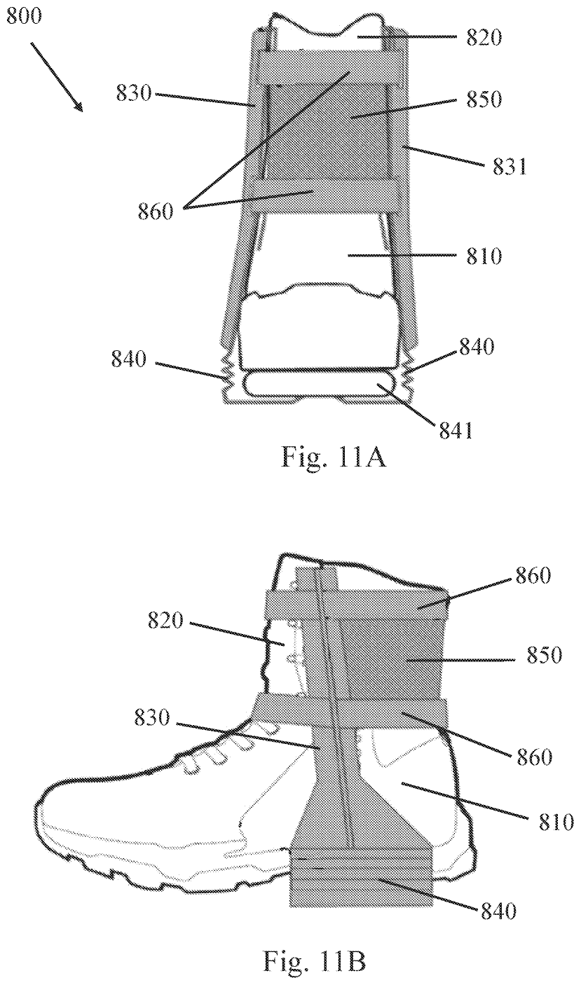

FIGS. 11A and 11B present schematic rear and side views, respectively, of a footwear system 800 having a lower portion 810 and an upper portion 820 of a footwear body. The footwear system also includes a fulcrum 840 and an incorporated shock absorber841. In some embodiments, the fulcrum 840 itself may serve to provide shock attenuation. As noted above, the incorporation of these elements 840 and 841 may serve to absorb and dissipate at least a portion of the applied shock and associated energy that may be incurred from a high amplitude landing force (e.g., due to landing on uneven ground), thereby preventing or reducing injuries to a lower limb of a user that may result from a landing impact force.

FIGS. 11A and 11B also show that the footwear system 800 may include a support sling comprising a pad 850, stirrups 860 configured to secure a user's lower limb, and one or more supports 830 and 831 (located on the medial and/or lateral sides) which may extend the length of the footwear body and be connected to the fulcrum 840.

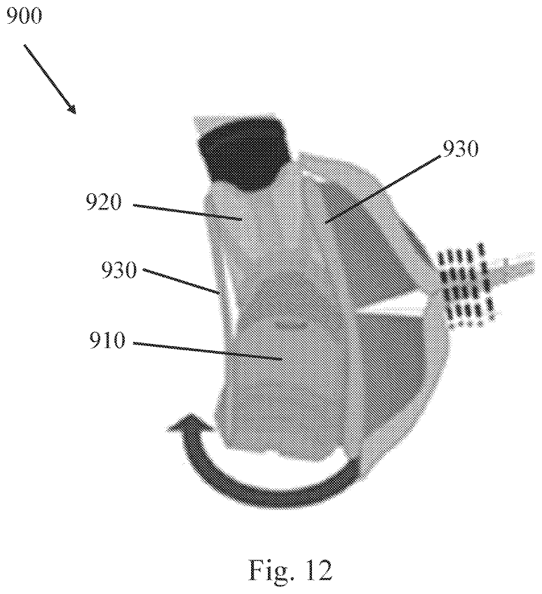

FIG. 12 presents a schematic rear view of a footwear system 900 including a lateral dampers 930 provided along the medial and lateral side of the footwear body such that the lateral dampers 930 extend between a lower portion 910 and an upper portion 920 of the footwear body. The inventors have found benefit to employing lateral dampers 930 in such an arrangement as they may provide support against inversion and/or eversion of a user's ankle. The lateral dampers 930 may include variable viscosity dampers or other friction dampers and rip straps or other suitable energy dissipating arrangements. These dampers may be secured to the footwear using any suitable arrangement including, for example, through stirrups which may affix the strap across the bottom of the heel to connect the strap between the medial and lateral sides of the footwear. Regardless of the orientation of the dampers, by traversing the dampers between portions of the footwear system, a larger radius and pathway for energy dissipation may be realized, thus helping to limit and/or prevent inversion and/or eversion injuries.

The embodiments described herein may be embodied as a method. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A footwear system comprising:
a footwear body;
a yoke that is configured to rotate relative to at least a portion of the footwear body in a sagittal plane in dorsiflexion and plantarflexion;
an energy storage device connected to the yoke and the portion of the footwear body; and
one or more supports configured to be engaged with the footwear body and/or the yoke,
wherein the one or more supports are configured to provide lateral support to an ankle of a user when the footwear system is worn by the user.

2. The footwear system of claim 1, wherein the footwear body and the yoke each have a medial side and a lateral side.

3. The footwear system of claim 2, wherein at least one of the one or more supports are configured to be located on the medial side of the footwear body and/or the medial side of the yoke.

4. The footwear system of claim 2, wherein at least one of the one or more supports are configured to be located on the lateral side of the footwear body and/or the lateral side of the yoke.

5.-6. (canceled)

7. The footwear system of claim 3, wherein the one or more supports is a singular support.

8. The footwear system of claim 3, wherein the one or more supports is a plurality of supports.

9. (canceled)

10. The footwear system of claim 1, wherein the yoke is configured to be removable from the footwear body.

11. (canceled)

12. The footwear system of claim 1, wherein the one or more supports are permanently retained in at least one of the footwear body and the yoke.

13. The footwear system of claim 1, further comprising one or more receptacles formed in the footwear body and/or the yoke, the one or more receptacles configured to removably receive the one or more supports.

14.-15. (canceled)

16. The footwear system of claim 13, where the yoke is configured to rotate relative to the portion of the footwear body at a pivot point, and wherein the one or more supports are configured to be positioned at variable distances relative to the pivot point.

17. The footwear system of claim 13, wherein the yoke is configured to rotate relative to the portion of the footwear body at a pivot point, and further comprising one or more connectors of a corresponding support of the one or more supports configured to engage the corresponding support received in a corresponding receptacle of the one or more receptacles at one of a plurality of predetermined positions relative to the pivot point.

18. The footwear system of claim 17, wherein the one or more connectors includes one or more detents formed in the one or more supports.

19. The footwear system of claim 1, wherein the one or more supports are configured to have a first lateral bending resistance in a first direction and a second lateral bending resistance in a second direction opposite to the first direction, wherein the first lateral bending resistance is different than the second lateral bending resistance.

20. The footwear system of claim 19, wherein at least one support of the one or more supports includes a corrugated surface extending along at least a portion of a length of the at least one support.

21. The footwear system of claim 1, wherein each support of the one or more supports includes a notch formed in a distal end of the support that is configured to be oriented towards the ankle of the user when the footwear system is worn by the user.

22. The footwear system of claim 1, wherein at least one support of the one or more supports includes a stiffening rib extending along at least a portion of a length of the at least one support.

23. The footwear system of claim 1, wherein a stiffness of the yoke is insufficient to support a force applied to the yoke by the energy storage device during use, and wherein a combined stiffness of the yoke and the one or more supports is sufficient to support the force applied to the yoke during use.

24. The footwear system of claim 1, wherein the energy storage device is selected from at least one of an elastic cord, a woven cord, a spring, an elastic band, a bungee cord, an elastic sheet, an elastic shaped component, a die-cut elastic component, and an elastic-reinforced fabric.

25. A method of using a footwear system comprising:
rotating a yoke relative to a portion of a footwear body during dorsiflexion and plantarflexion of a user's ankle;
storing and/or releasing energy with an energy storage device during the relative rotation of the yoke and the portion of the footwear to assist motion of the user's ankle; and
engaging one or more supports with the footwear body and/or the yoke to provide lateral support to the user's ankle.

26. The method of claim 25, wherein the footwear body and the yoke each have a medial side and a lateral side.

27. The method of claim 26, wherein engaging the one or more supports with the footwear body and/or the yoke includes engaging at least one of the one or more supports with the medial side of the footwear body and/or a medial side of the yoke.

28. The method of claim 26, wherein engaging the one or more supports with the footwear body and/or the yoke includes engaging at least one of the one or more supports with the lateral side of the footwear body and/or a lateral side of the yoke.

29.-36. (canceled)

37. The method of claim 25, further comprising disengaging the one or more supports and engaging one or more separate supports with the footwear body and/or the yoke.

38. The method of claim 25, wherein engaging the one or more supports with the footwear body and/or the yoke includes inserting the one or more supports into one or more receptacles.

39.-40. (canceled)

41. The method of claim 38, wherein the yoke is configured to rotate relative to the portion of the footwear body at a pivot point, and further comprising positioning the one or more supports at variable distances relative to the pivot point within a corresponding receptacle of the one or more receptacles.

42.-43. (canceled)

44. The method of claim 25, wherein the one or more supports are configured to have a first lateral bending resistance in a first direction and a second lateral bending resistance in a second direction opposite to the first direction, wherein the first lateral bending resistance is different than the second lateral bending resistance.

45. The method of claim 44, wherein at least one support of the one or more supports includes a corrugated structure extending along at least a portion of a length of the at least one support.

46. (canceled)

47. The method of claim 25, wherein at least one support of the one or more supports includes a stiffening rib extending along at least a portion of a length of the at least one support.

48.-49. (canceled)

50. A restorative energy system for footwear, the system comprising:
a base configured to be engaged with a first portion of a footwear body;
a yoke configured to be engaged with a second portion of the footwear body, wherein the yoke is configured to rotate relative to the base and the first portion of the footwear body;
an energy storage device connected to the base and the yoke; and
one or more supports configured to be attached to the base and/or the yoke, wherein the one or more supports are configured to provide lateral support to an ankle of a user.

51.-73. (canceled)

74. A method of using a restorative energy system for footwear, the method comprising:
engaging a base with a first portion of a footwear body;
engaging a yoke to a second portion of the footwear body, the yoke configured to rotate relative to the base and the first portion of the footwear body;
connecting an energy storage device to the base and the yoke,
storing and/or releasing energy with the energy storage device during the rotation of the yoke relative to the base and the first portion of the footwear body;
engaging one or more supports with the base and/or the yoke to provide lateral support to an ankle of a user.

75.-96. (canceled)
